(19) United States
(12) Patent Application Publication
(10) Pub. No.: US 2026/0314927 A1
(43) Pub. Date: Oct. 15, 2026

(54) RACIALLY FAIR PUPILLOMETRY MEASUREMENTS FOR RGB SMARTPHONE CAMERAS USING THE FAR RED SPECTRUM

(71) Applicant: The Regents of the University of California, Oakland, CA (US)

(72) Inventors: Edward Jay Wang, La Jolla, CA (US); Colin Barry, La Jolla, CA (US)

(21) Appl. No.: 19/486,850

(22) PCT Filed: May 22, 2024
(86) PCT No.: PCT/US2024/030635

§ 371 Date: Nov. 21, 2025

Related U.S. Application Data
(60) Provisional application No. 63/503,935, filed on May 23, 2023.

(51) Int. Cl. A61B 3/11; A61B 3/14; A61B 5/00

(52) U.S. Cl. CPC A61B 3/112; A61B 3/14; A61B 5/6898

(57) ABSTRACT

A portable pupillometer device is disclosed in this disclosure. The portable pupillometer device includes an electronic device having a frontside surface and a rear surface, the electronic device comprising one or more RGB cameras disposed on the frontside surface of the electronic device, a light source, and a pupillometer attachment disposed on the frontside surface of the electronic device, including a case, a long-pass far red spectrum filter disposed in the case, the long-pass far red spectrum filter is configured to pass incident light in a far red spectrum ranging from 630 nm to 700 nm, and an internal opening disposed between the light source and the long-pass far red spectrum, wherein the light source is aligned internally to a periphery of the internal opening to allow incident light to be emitted through the long-pass far red spectrum.

### CROSS-REFERENCE TO RELATED APPLICATION(S)

The present international application claims priority to U.S. Provisional Patent Application No. 63/503,935, filed on May 23, 2023. The entire contents of the above noted provisional application are incorporated by reference as part of the disclosure of this document.

### STATEMENT REGARDING FEDERALLY SPONSORED RESEARCH OR DEVELOPMENT

This invention was made with Government support under grant no. 1R21AG072534 awarded by the National Institute of Aging. The Government has certain rights in the invention.

### TECHNICAL FIELD

The present disclosure generally relates to pupillometry measurement and more particularly relates to racially fair pupillometry measurements using RGB smartphone cameras and far red spectrum light.

### BACKGROUND

Pupillometry is a measurement of pupil dilation and is frequently conducted during neurological assessments to provide insights into neurological functions in response to various stimuli such as light, sound, and emotional triggers. This method has proven useful in diagnosing and monitoring a range of neurological disorders, including Alzheimer's Disease, Schizophrenia, Traumatic Brain Injury, Parkinson's Disease, opioid use, and Mild Cognitive Impairment, serving as a valuable quantitative biomarker for conditions that are otherwise challenging to measure directly. Currently, the widespread adoption of pupillometry is hindered by the high cost and inaccessibility of specialized pupilometers that operate in the near-infrared spectrum, typically available only in well-resourced clinics. There is a notable potential for extending this measurement to more accessible devices like smartphones or tablets equipped with RGB cameras, although challenges remain due to the melanin in the iris absorbing light in the visible spectrum, which makes distinguishing the black pupil aperture difficult.

### SUMMARY

A portable pupillometer device is described in the present disclosure. In some implementations, the portable pupillometer device includes an electronic device having a frontside surface and a rear surface, the electronic device including one or more RGB cameras disposed on the frontside surface of the electronic device; a light source; and a pupillometer attachment disposed on the frontside surface of the electronic device, including a case, and a long-pass far red spectrum filter disposed in the case, the long-pass far red spectrum filter is configured to pass light in a far red spectrum ranging from 630 nm to 700 nm, wherein the light source is configured to emit light to a user's eye and the long-pass far red spectrum filter is configured to pass light that is reflected from the user's eye and that has a wavelength falling in the far red spectrum.

Implementations can include one or more of the following features. For example, in some implementations, the electronic device is a smartphone. In some other implementations, the light source is a screen of the smartphone. In addition, the light source is a flashlight of the smartphone, the flashlight being disposed on the frontside surface or the rear surface of the smartphone. In some implementations, the pupillometer attachment further includes a neutral density filter that is disposed in the case and that is configured to lessen an intensity of the flashlight. In some other implementations, the pupillometer attachment further includes a macro lens that is disposed above a top surface of the long-pass far red spectrum filter. In particularly, the macro lens has a focal length ranging from 1 mm to 50 mm and a magnification ranging from 5 to 30. In some implementations, the light source is disposed on a frontside surface of the PCB. Additionally, the light source includes one or more light emitting diodes (LEDs) configured to emitting light with wavelengths ranging from 630 to 700 nm, the one or more LEDs being disposed on a frontside surface of the PCB. In some implementations, the portable pupillometer device further includes a power source disposed on a backside surface of the PCB. In some implementations, the pupillometer attachment further includes an eye cup assembled with the case; and a mounting structure including one or more screw caps and one or more fastening screws, where in the mounting structure is attached to the case and is configured to fastening the pupillometer attachment to the electronic device.

In another general aspect, the portable pupillometer device includes an electronic device including one or more RGB cameras disposed on a surface of the electronic device; and a pupillometer attachment assembled to the surface of the electronic device, the pupillometer attachment covers the one or more RGB cameras and includes a case, a custom circular PCB that is disposed in the case, the PCB having a profile that allows lights passing through, and one or more light emitting diodes (LEDs) disposed on a frontside surface of the PCB, the LEDs emitting light with wavelengths ranging from 630 to 700 nm, wherein the one or more LEDs are configured to emit a far red spectrum light with a wavelength ranging from 630 nm to 700 nm to a user's eye, and the one or more RGB cameras captures reflected far red spectrum light from the user's eye.

In some implementations, the portable pupillometer device further includes a macro lens that is disposed at a center of the PCB, the macro lens having a focal length ranging from 1 mm to 50 mm and a magnification ranging from 5 to 30. In some other implementations, the pupillometer attachment further includes an eye cup assembled with the case; and a mounting structure including one or more screw caps and one or more fastening screws, where in the mounting structure is attached to the case and is configured to fastening the pupillometer attachment to the electronic device.

In another general aspect, a method of operating a portable pupillometer device is described in this disclosure. The method of operating a portable pupillometer device includes assembling a pupillometer attachment with an electronic device; powering on, through operating a pupillometer interface software on the electronic device, the pupillometer attachment; placing the pupillometer attachment above a user's eye; emitting incident light to the user's eye; capturing, by one or more RGB cameras of the electronic device, light reflected from the user's eye; and generating user iris images or videos on a screen of the electronic device.

In some implementations, the assembling the pupillometer attachment with the electronic device includes fastening, by using a mounting structure, the pupillometer attachment to the electronic device, and covering the one or more RGB cameras of the electronic device by the pupillometer attachment. In some implementations, emitting incident light includes stimulating a flashlight of the electronic device, and wherein the capturing of light reflected from the user's eye includes filtering or passing reflected light with wavelengths ranging from 630 nm to 700 nm by a long-pass far red spectrum filter disposed in the pupillometer attachment. In some implementations, emitting the incident light includes emitting, by one or more light emitting diodes (LEDs) of the pupillometer attachment, a far red light with a wavelength ranging from 630 nm to 700 nm by one or more LEDs included in the pupillometer attachment, and wherein capturing the light reflected from the user's eye includes capturing the far red light reflected from the user's eye by the one or more RGB cameras of the electronic device. In some other implementations, the method of operating the portable pupillometer device further includes generating user pupil diameter and position values through analyzing the user iris eye images or videos by one or more convolution neural network (CNN).

### BRIEF DESCRIPTION OF THE DRAWINGS

FIG. 1A illustrates an overview of a portable pupillometer device with commodity smartphone cameras in accordance with various embodiments of the present technology.

FIG. 1B shows light iris and dark iris images collected from a full spectrum Digital Single-Lens Reflex (DSLR) camera and the portable pupillometer device.

FIG. 2 shows images of a same dark iris eye taken in various light spectrums.

FIG. 3 shows a far-red pupillometer utilizes an IR-pass filter that covers a standard silicon-based camera to expose the far-red spectrum, in accordance with various embodiments of the present technology.

FIG. 4A illustrates a fully assembled pupillometer attachment module in accordance with various embodiments of the present technology.

FIG. 4B illustrates individual components included in the pupillometer attachment module in accordance with various embodiments of the present technology.

FIG. 5 shows pupil diameter comparison of pupillary light reflex responses recorded with the portable pupillometer device and Neuroptics pupillometer, in accordance with various embodiments of the present technology.

FIGS. 6A and 6B show a Regression and Bland Altman plots of pupil diameter results collected by the portable pupillometer device and Neuroptics pupillometer, respectively in accordance with various embodiments of the present technology.

FIG. 7 is a flow chart illustrating a method for operating a portable pupillometer device in accordance with various embodiments of the present technology.

### DETAILED DESCRIPTION

The present technology aims to facilitate the use of pupillometry and related research areas through vastly expanding access to pupil dilation measurements. Currently, the multi-thousand dollar cost of pupillometers restricts the use of pupillometry outside of high resource institutions even though, pupillometry has significant potential beyond clinical laboratories. It has been demonstrated that pupillometry has potential for prognostic assessments from acute head injuries, at-home cognitive screening in older adults, or opioid testing. However, the lack of pupillometry resources contributes to a lack of research on at-home pupillometry tests, test retest reliability studies of novel pupil biomarkers, and high temporal resolution studies of multi-day or multi-week studies. To expand pupillometry access, the present technology provides a novel method of enabling everyday smartphone RGB cameras to perform pupillometry measurements.

In addition, the present technology seeks to democratize access to pupillometry and enhance its application in diverse research fields by making pupil dilation measurements more widely available. Traditionally, the utilization of pupillometry has been confined primarily to well-funded institutions due to the prohibitive costs associated with pupilometers, which can reach several thousands of dollars. Despite its substantial potential for prognostic evaluations in cases of acute head injuries, at-home cognitive assessments in elderly populations, and opioid use testing, the accessibility of pupillometry remains limited. This restriction not only impedes clinical usage but also stifles research advancements in areas such as at-home pupillometry testing, the reliability of novel pupil biomarkers, and high temporal resolution studies spanning multiple days or weeks. In response to these challenges, the present technology introduces an innovative method that enables conventional smartphone RGB cameras to conduct pupillometry measurements, thereby expanding the technology's reach and utility.

The efficacy of pupillometry hinges on the ability to clearly distinguish the pupil from the iris in captured images. In visible light, particularly for individuals with darker irises, differentiating between the pupil and iris proves challenging. The pupil, functioning as an aperture, allows light to reach the eye's interior where it is largely absorbed, rendering the pupil visibly black. Traditional clinical pupillometers and high-performance systems employ near-infrared (NIR) light to enhance pupil measurement, taking advantage of the iris's reflection of longer wavelengths, which appear bright and create a stark contrast with the dark pupil. However, NIR imaging is impractical with standard smartphone RGB cameras, which incorporate filters that block light outside the visible spectrum to enhance photographic quality. Consequently, existing commercial pupillometry technologies cannot consistently operate across different smartphone models, nor can they adequately improve the contrast between pupil and iris in RGB images. While there are studies utilizing convolutional neural network (CNN) to segment the pupil from the iris in smartphone-based pupillometry, these efforts are inherently constrained by the low contrast between the pupil and iris in smartphone camera images. The present technology innovatively addresses the challenge of executing pupillometry within the limited light spectrum available to common RGB cameras. Specifically, the present technology leverages the ubiquity of smartphone cameras to facilitate widespread pupillometry applications by both individuals and consumers, enhancing accessibility and utility.

The present technology also addresses the challenge of ubiquitous smartphone-based pupillometry, designed to function effectively across a diverse range of smartphone devices, which vary in components, interfaces, and physical designs. This universal applicability is essential for enabling broad access to pupillometry applications, ranging from high school sports coaches assessing potential concussions in students to facilitating neurological screenings in older adults. The present technology leverages existing smartphone hardware and software to deliver an inexpensive solution for pupillometric measurements. Importantly, the system(s) of the present technology can be engineered to perform optimally on a vast array of smartphones, including lower-cost models.

A critical aspect of the present technology is its enhanced capability to accurately segment the pupil from the iris in individuals with dark-colored irises, specifically dark brown, under various lighting conditions. Previous attempts in smartphone-based pupillometry often excluded individuals with dark iris colors or reported suboptimal performance due to the difficulty in distinguishing the pupil from the iris using conventional imaging methods within the full visible spectrum. The present technology overcomes these limitations by employing advanced CNN, which are meticulously trained to improve the accuracy of pupil detection in challenging scenarios, thereby ensuring inclusivity and reliability in pupillometric assessments across all demographics.

There are reported attempts at implementing accurate pupillometry through the use of Near Infrared (NIR) facial recognition cameras in smartphones. Those attempts have shown promise but are limited by hardware availability. Specifically, NIR cameras have been predominantly featured only in higher-end smartphone models and are currently being phased out by major manufacturers, such as Google and Motorola. Furthermore, even in devices that do include NIR cameras, manufacturers generally restrict access to these cameras, preventing developers from utilizing them for third-party applications such as pupillometry. Those reported attempts therefore fails to provide a solution that can be universally applied across the wide spectrum of available smartphone devices. In comparison, the present technology introduces a novel methodology for conducting smartphone-based pupillometry that effectively enhances the contrast between the pupil and the iris. The present technology requires only the presence of one or more RGB cameras, which are components ubiquitously included in all major smartphone brands, thus ensuring a wide applicability. Unlike previous attempts that depended on specialized hardware, the present technology leverages common smartphone technology to provide an accurate and accessible pupillometric assessment, thereby overcoming the limitations of prior works and broadening the potential user base.

In the present technology, a novel pupillometry technique is developed to enable smartphone RGB cameras to effectively differentiate the pupil from the iris. The present technology includes a pupillometry attachment which can be attached on a smartphone to form a portable pupillometer system. This pupillometer system can utilize a 630 nm long-pass filter to image in the far red (630 nm-700 nm) spectrum, where the melanin in the iris reflects light to appear brighter in contrast to the dark pupil. Alternatively, the portable pupillometer system can utilize a light source to emit the far red spectrum light, e.g., light emitting diodes (LEDs) embedded in the pupillometry attachment, for pupillometry testing. Far red spectrum light reflected from a user's eye can be captured by smartphone RGB cameras and form iris eye images or videos. Using a CNN, the portable pupillometer system can measure pupil diameter as it dynamically changes in a frame by frame video. Comparing across 4 different smartphone models, the pupil-iris contrast of N=12 participants increases by an average of 451% with the proposed system. In a validation study of N=11 participants comparing the relative pupil change in the proposed system to a Neuroptics PLR-3000 Pupillometer during a pupillary light response test, the prototype portable pupillometer system of the present technology achieved a mean absolute error of 2.4%.

Preliminary studies of the present technology involves a custom prototype portable pupillometer device which demonstrated the potential of the disclosed novel methodology to significantly broaden the accessibility of pupillometry. Here, the objective is to facilitate the deployment of pupillometry-based testing for detecting concussions, drug usage, and other neurological conditions not only in clinical settings but also in homes, schools, and sporting events. The present technology employs filtered far-red wavelengths within the visible spectrum, which are specifically chosen to enhance the distinction between the pupil and the iris when imaged with RGB cameras.

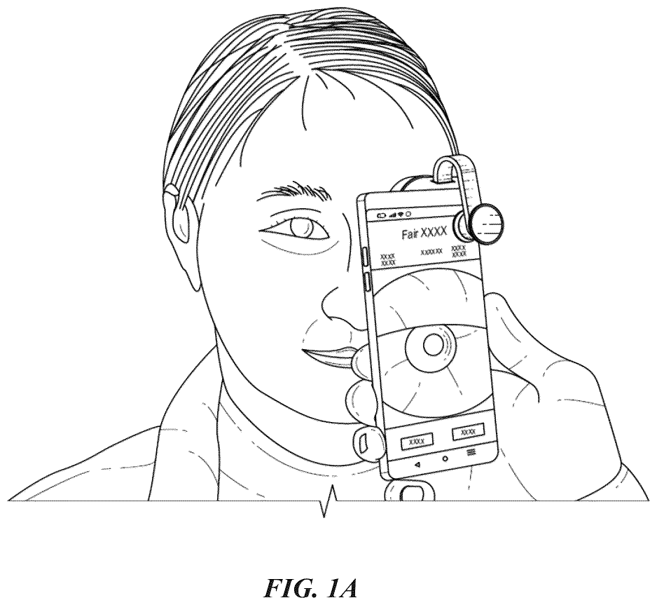

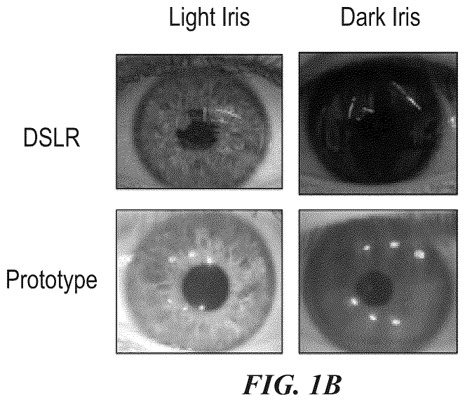

FIG. 1 demonstrates an overview of a pupillometry system utilizing commodity smartphone cameras. Specifically, FIG. 1A illustrates collecting a user's iris image by using a portable pupillometer device involving commodity smartphone cameras in accordance with various embodiments of the present technology. In this example, a conventional smartphone can be equipped with a pupillometer attachment module, thereby constructing a portable pupillometry device. As illustrated, this portable pupillometry device can either be manually held by a user or attached to a supporting structure to facilitate the collection of iris images or videos. This configuration allows for enhanced flexibility and mobility in the application of pupillometry technologies.

In this example, a custom smartphone application is developed and utilized to administer pupil response test stimuli while simultaneously recording the pupil response. The goal of the smartphone application is to enable a user to perform a pupil response tests on other individuals. As shown in FIG. 1, this application includes a camera preview of the eye to position the smartphone correctly before starting the tests. The user is meant to position the pupil clearly within the camera frame with the scope touching the test participant's face. To facilitate single hand use of the smartphone for test administration, the physical volume buttons on the side of the smartphone can be used to start a pupil response test. During a pupil response test, the smartphone application displays real time video with a testing in progress label on the on-screen buttons.

In the present disclosure, the term “far red light spectrum” refers to the longer wavelengths of red light within the visible spectrum at approximately 630 nm to 700 nm. The present technology utilizes the far red light spectrum for pupillometry applications and to convey several advantages traditionally associated with infrared, clinical pupillometers to more accessible devices, including commercial cameras and smartphones. FIG. 1B show light iris and dark iris images collected from a full spectrum DSLR camera and the prototype portable pupillometer device, respectively. In comparison to the iris images collected from DSLR camera, it could be found that the light iris and dark iris images generated from the prototype far red light spectrum significantly improve the ability to distinguish the pupil in the iris images.

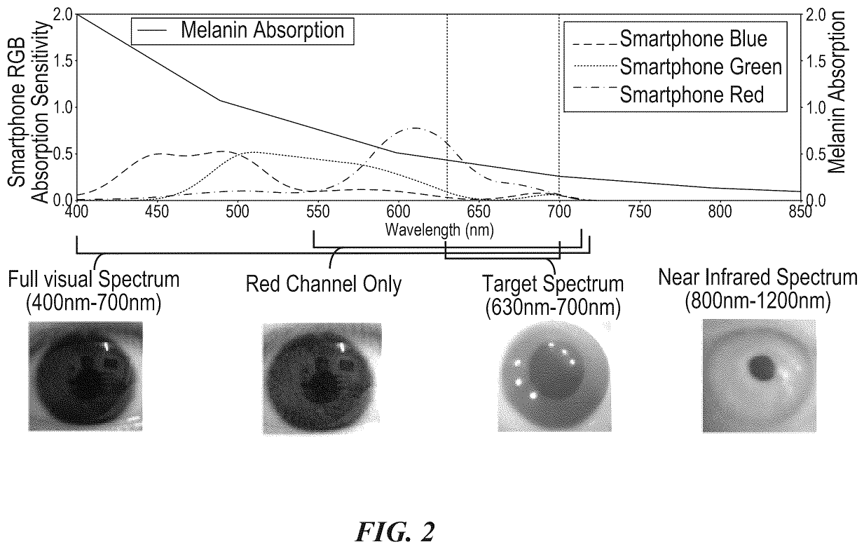

FIG. 2 shows images of a same dark iris eye taken in various light spectrums. In this example, the light spectrum illustrate on top can be categorized into a full visual spectrum (400 nm-700 nm), red channel (approx. 550 nm-700 nm), far red/target light spectrum (630 nm-700 nm), and a near-infrared spectrum (approx. 800 nm-1200 nm). In addition, there are four iris images taken on the same dark iris eye using different light spectrums and shown in the bottom. These iris images are included here to allow a user to see the difference in pupil to iris contrast in different imaging conditions. Specifically, the imaging spectrums include the full visual spectrum and red channel spectrum are both used by a DSLR RGB camera. The target far red spectrum (630 nm-700 nm) utilized in the present technology is identified with vertical dotted lines. The light spectrum of FIG. 2 also discloses melanin absorption at different wavelengths which is plotted in solid black. Additionally, the absorption of the red, green, and blue channels of a smartphone camera has been reported to exemplify the absorption of the red and full visible spectrum in smartphone cameras.

In the present technology, the novel pupillometry method is implemented in a prototype pupillometer attachment containing LEDs and a long-pass far red spectrum filter to ensure the images contain only the target spectrum of approximately 630 nm-700 nm. The prototype portable pupillometer device leverages the smartphone camera, flashlight LED, and a custom application to perform a pupillary light reflex test. With the pupillometer attachment module assembled, the smartphone records the participant's pupil response as the smartphone flashlight provides a bright light stimulus. The functional prototype demonstrates that this filtered spectrum is suitable for pupillometry and achievable on nearly all commodity smartphone RGB cameras.

The present technology utilizes the far red edge of the visible spectrum to conduct pupillometry. A primary objective of this prototype pupillometer attachment module is to enable conventional smartphones to exclusively capture images within the far red wavelength spectrum (approximately 630 nm-700 nm). In this spectrum range, located at the periphery of human visible light, the absorption characteristics of the pupil and iris differ significantly. Most standard RGB camera filters employed in commercial smartphones can transmit this far red light. Notably, melanin exhibits substantially reduced absorption in this far red spectrum compared to other parts of the visible spectrum, resulting in a much lighter appearance of the iris, particularly for individuals with high melanin content and dark brown irises. Typically, the red channel of a smartphone's camera is broadband, capturing a wide range of wavelengths, where shorter wavelengths are absorbed, leading to diminished contrast between the pupil and iris. The integration of a long-pass filter in the pupillometer attachment module restricts imaging to the desired wavelength range, enhancing the contrast necessary to distinguish the pupil from the iris effectively, as demonstrated in FIG. 2.

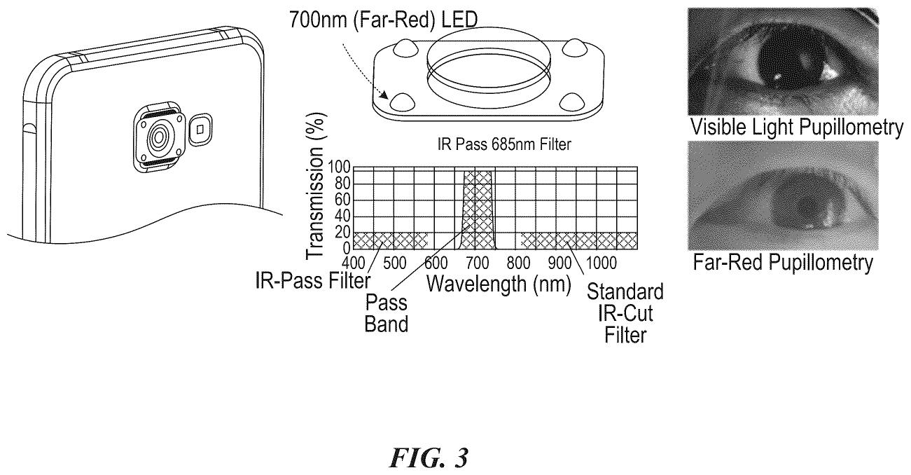

FIG. 3 shows a far-red pupillometer utilizing an IR-pass filter that covers a standard silicon-based camera to expose the far-red spectrum, in accordance with various embodiments of the present technology. In this example, an IR-pass through filter is placed with a cut-off at 685 nm, which is a standard off-the-shelf wavelength used by hobbyist planetary photography. Combining this IR-pass filter with the IR-cut filter in color cameras creates a passband there between that can capture the eye with little influence from melanin. By illuminating the eye with a 700 nm Far Red LED, the far-red pupillometer shown in FIG. 3 can capture dark-colored eyes while not being strongly visible to the eye as to affect the actual pupillometry effort. In comparison of the visible light pupillometry image and the far-red pupillometry image, it can be found that the far-red spectrum light can better distinguish the iris from the pupil.

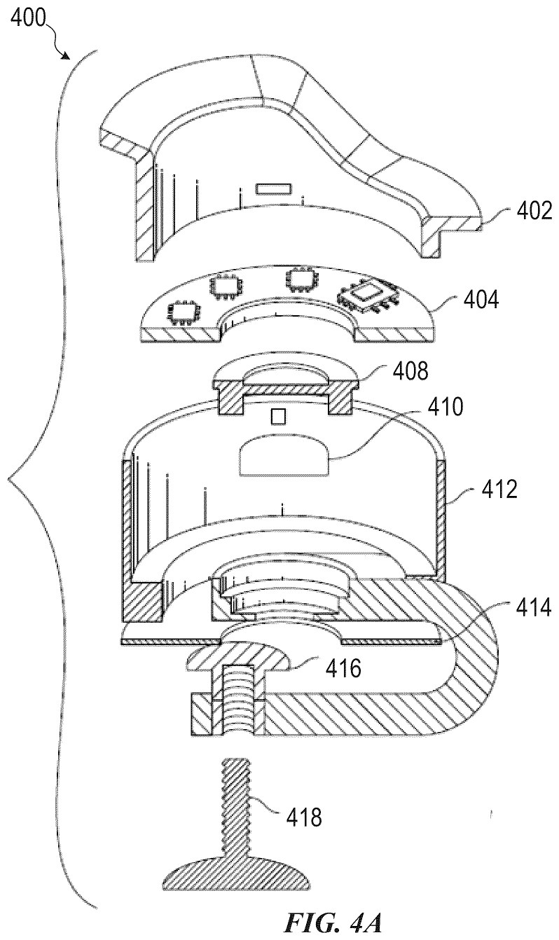

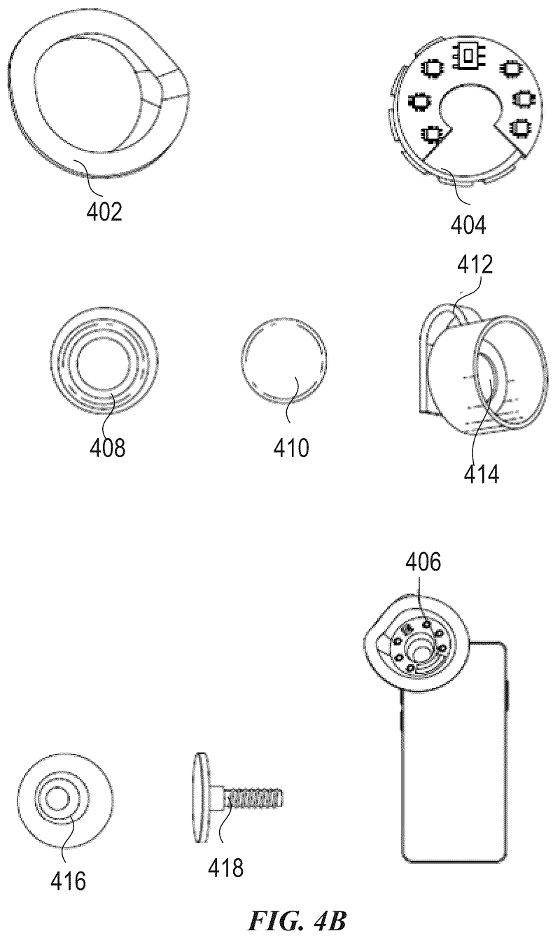

The present technology includes to a universal pupillometer attachment module designed to adapt to the majority of contemporary smartphones, provided that the back camera is positioned within 2.5 cm from the edge of the device. FIG. 4A illustrates a fully assembled pupillometer attachment module 400 in accordance with various embodiments of the present technology. This pupillometer attachment module 400 accommodates various layouts of the camera and flash LED, ensuring compatibility across different smartphone models. In particular, the pupillometer attachment module 400 is enclosed within a 3D-printed circular case 412. Specifically, the prototype pupillometer attachment module 400 comprises four essential elements: a 630 nm long-pass filter 410 (e.g., an Edmund Optics SCHOTT RG-630, 12.5 mm Diameter, 3 mm Thick Colored Glass Long-pass Filter), a custom circular PCB 404 equipped with 625 nm LEDs (Marubeni 625SMT LEDs) and one one/off switch, a 15× macro lens 408, and a 3D-printed rubber eye cup 402. In this example, the prototype pupillometer attachment module 400 also comprises a neutral density filter 414, and a mounting structure including one or more screw caps 416 and one or more fastening screws 418. As shown in FIG. 4A, the long-pass filter 410, positioned at the rear of the pupillometer attachment module 400, is strategically placed to align directly in front of the smartphone's camera lens when attached, blocking light wavelengths shorter than 630 nm while allowing those in the target spectrum of approximately 630 nm-700 nm to pass through. This configuration effectively transforms a standard smartphone camera into a specialized pupillometry device capable of imaging in the specified wavelength range.

In this example, the macro lens 408 incorporated into the pupillometer attachment module 400 is configured to adjust the camera's focus to magnify the pupil, enabling precise measurement of its size at close range. This prototype design does not rely on specific ambient lighting conditions, as it generates an appropriate lighting environment using six Marubeni SMT 625 nm LEDs, all with a spectral half-width of 25 nm. These LEDs can be powered by a button cell battery and controlled via a simple switch located on the PCB 404, which is shaped as a semi-circle to facilitate positioning over the smartphone's flashlight 406, assuming it is approximately 1.5 cm from the camera. In this example, the smartphone's flashlight 406 can act as a stimulus to trigger pupil constriction, managed by a smartphone application according to standard pupillary response test protocols. The neutral density filter 414 is also integrated at the back of the 3D-printed case 412 to cover the smartphone flashlight 406, allowing the LEDs to safely illuminate the participant's eye without causing excessive light exposure. Furthermore, the rubber eye cup 402 serves to block extraneous environmental light, preventing unwanted stimuli or glare during the measurement process. This pupillometer attachment module 400 ensures precise positioning of the camera in 3D space to focus accurately on and measure the pupil, enhancing the usability and effectiveness of the pupillometer.

In this example, the pupillometer attachment module 400 may include an internal opening disposed between the smartphone's flashlight 406 and the long-pass far red spectrum filter 410. Particularly, the smartphone's flashlight 406 can be aligned internally to a periphery of the internal opening in order to allow incident light to be emitted through the long-pass far red spectrum filter 410. Here, the smartphone's flashlight 406 is configured to emit light to a direction of a user's eye through the long-pass far red spectrum filter 410, and the long-pass far red spectrum filter 410 is configured to pass reflected light that is reflected from the user's eye and that has a wavelength falling in the far red spectrum. In addition, smartphone cameras can be aligned with a center of the internal opening to allow reflected light to be received by the one or more RGB cameras through the center of the internal opening.

FIG. 4B illustrates individual components included in the pupillometer attachment module 400 in accordance with various embodiments of the present technology. In this example, the 3D-printed case 412 is utilized to house the components of the pupillometer attachment module 400, providing structural support and aligning them correctly with the smartphone camera. As described in FIG. 4A, the rubber eye cup 402 is disposed on a frontside surface of the case 412, ensuring contact with the user's face and blocking extraneous light and stabilizing the pupillometer attachment module 400 during measurement. The custom-designed, semi-circular PCB 404 is outfitted with 625 nm LEDs to provide the necessary spectral illumination for pupillometry. In this example, the macro lens 408 is disposed on the PCB 404 to allow the smartphone's camera to focus closely on the eye, enabling accurate measurement of the pupil's size. The long-pass filter 410 can be disposed under the PCB 404 and permits light above 630 nm to pass through while blocking shorter wavelengths. As shown, the scree cap 416 and screw 418 can be disposed at a bottom surface of the case 412, to secure the neutral density filters in place with the case 412 and attach the pupillometer attachment module 400 firmly to a smartphone.

In another embodiment, the pupillometer attachment module 400 can be composed by a long-pass filter and a light source for the purpose of pupillometry. For example, the long-pass filter 410 disposed within the case 412 and the smartphone flashlight from the smartphone can be used to form the pupillometer device. Specifically, the 3D-printed case 412 can be utilized to house the components of the pupillometer attachment module 400, including the eye cup 402, the macro lens 408, the long-pass filter 410, the neutral density filter 414, and the fastening attachments including the screw cap 416 and the screw 418. In this example, the pupillometer attachment module 400 can be assembled on a surface of the smartphone having one or more RGB cameras and a flashlight. In another example, the pupillometer attachment module 400 can be assembled on a frontside surface of the smartphone, utilizing a frontside RGB camera and the screen as light source for pupillometry. Here, the light emitting to and/or reflecting from the test participant's eye are filtered by the long-pass filter 410. In particularly, the reflected far red spectrum light can pass through the center and/or periphery of the PCB 404, and collected by the one or more RGB cameras of the smartphone.

As a result, only far red spectrum light reflected from the test participant's eye is collected by the RGB camera, in assisting the pupil measurement.

In some other embodiments, the pupillometer attachment module 400 can be composed of Far Red LEDs emitting light with wavelength ranging from 630 nm to 700 nm. In this example, the 3D-printed case 412 can be utilized to house the components of the pupillometer attachment module 400, including the eye cup 402, the PCB 404 with Far Red LEDs disposed thereon, the macro lens 408, and the fastening attachments including the screw cap 416 and the screw 418. Here, the pupillometer attachment module 400 can be attached on a backside of a smartphone to isolate light emitting from the smartphone. Moreover, the Far Red LEDs disposed on the PCB 404 is configured as the light source to emitting far red spectrum light to a test participant's eye. As a result, the far red spectrum light will be reflected from the test participant's eye and collected by RGB cameras of the smartphone. In particular, the reflected far red spectrum light can pass through the center and/or periphery of the PCB 404, and collected by the smartphone.

In validating the accuracy of the pupillometry involving the pupillometer attachment module 400 and smartphone cameras, pupillary light reflex tests are performed. Pupillary light reflex (PLR) tests involve stimulating the pupil with bright light to evoke a large pupillary constriction. The PLR test is chosen because it provides a common and valuable biomarker with significant potential for neurological screening, especially for traumatic brain injury (TBI) test. Here, the PLR light stimulus can be provided by the smartphone-based prototype by using an application that controls the smartphone flashlight. The pupillometer attachment module 400 contains a neutral density filter 414 to lessen the intensity of the smartphone flashlight 406. Pupil measurement of N=12 participants with the prototype is recorded simultaneously with a clinical pupillometer as a ground truth measurement. The validation results in a mean absolute error (MAE) of 2.4%, which improves upon the current state of the art for smartphone pupillometry.

In the validation experiment, the smartphone-based portable pupillometry device is compared to the Neuroptics PLR 3000. For the study comparison, the smartphone system records from the left eye, while the Neuroptics device simultaneously records from the right eye. The Neuroptics device records absolute pupil size in millimeters at 30 Hz while the prototype smartphone system records pupil size in pixels at 60 Hz. The measurements are aligned in time by down sampling the smartphone data points to 30 Hz and excluding blinks from both devices. The two measurements are then compared using percent change from mean. The Neuroptics device does not have the capacity to be controlled remotely so the recordings are started by simultaneous button press. As such, the recordings starts can be misaligned, so some recordings are manually aligned in post-processing. In this example, both devices record for exactly 5 second. While the smartphone administers a 5 second PLR test, the Neuroptics device records, but provides no stimulus. The smartphone provides the only stimulus during the test.

The PLR test involves a 5 second recording with a short light stimulus. In this example, the PLR test protocol can be configured as follows: 1 second of no light, 1 second of light stimulus provided by the camera flashlight, 3 seconds of no light. Note that the smartphone flashlight LED is covered by the neutral density filter of the prototype to avoid excessive brightness in the light stimulus. During the entirety of the PLR test, the far red LEDs of the prototype illuminate the eye for imaging. No other sound or stimuli are included in the study.

The PLR test is chosen due to its application and versatility in pupillometry. Researchers and clinicians commonly use this test to investigate a number of neurological conditions including traumatic brain injury, cognitive impairment, and substance use. The PLR test within the validation experiment demonstrates functionality across a range of pupil sizes and provides a comparable measurement to prior work.

The generalizability of the pupillometry utilizing the present technology is assessed across 4 smartphone models of different manufacturers. It has been demonstrated that the prototype plastic pupillometer attachment module with LEDs and light filters can be attached to all tested smartphones to enable accurate pupillometry measurements. On each smartphone model, video frames of (N=11) pupils taken in the far red lighting condition are compared to a normal lighting condition. The pupil to iris contrast yields an average 451% increase across the smartphones with the far red lighting.

In this example, participants were recruited to have a bias towards darker eye colors because darker eye colors are more difficult to differentiate from the pupil in the visible spectrum based on prior work. The participant eye color is defined qualitatively by the participant's self-assessment of their eye color in the following categories: light/blue, hazel/light brown, and dark brown. The eye color is also quantitatively characterized by the iris luminance based on an image from a DSLR camera (e.g., Camera: Cannon EOS R6 Mark II, Lens: Cannon RF 35 mm). The DSLR camera provides a single, high quality image for analysis. Within an image, a 15×15 pixel area of the iris is manually selected for analysis. The median luminance of the selected iris pixels are reported for iris. Luminance is calculated by converting the RGB image to the CIELAB color space to quantify iris color as similarly performed in prior work. The participant demographics and eye colors involved in this work are available in Table 1. Participants 11 and 13 did not participate in the cross device compatibility study. In addition, participant 12 was excluded from the validation study because of erroneous measurements in the ground truth neuroptics device.

TABLE 1
Participant demographics.

| Participant | | | | Eye color | Iris |
|---|---|---|---|---|---|
| ID | Gender | Age | Ethnicity | description | luminance |
| 1 | M | 26 | White | Blue | 116 |
| 2 | M | 25 | White | Blue | 104 |
| 3 | M | 26 | White | Blue | 84 |
| 4 | F | 28 | Hispanic/ | Light brown | 63 |
| | | | Latino | | |
| 5 | M | 24 | Asian | Dark brown | 55 |
| 6 | M | 26 | Asian | Dark brown | 45 |
| 7 | M | 25 | Asian | Dark brown | 42 |
| 8 | M | 28 | Asian | Dark brown | 36 |
| 9 | M | 21 | Asian | Dark brown | 35 |
| 10 | M | 29 | Asian | Dark brown | 34 |
| 11 | F | 30 | Asian | Dark brown | 31 |
| 12 | F | 23 | Asian | Dark brown | 26 |
| 13 | M | 23 | Asian | Dark brown | 18 |

Note that smartphone videos and images were specifically avoided for this measurement. The computation photography integrated in smartphones can significantly alter image color by increasing local and/or global dynamic range. The DSLR camera is preferable over smartphone videos for the image quality.

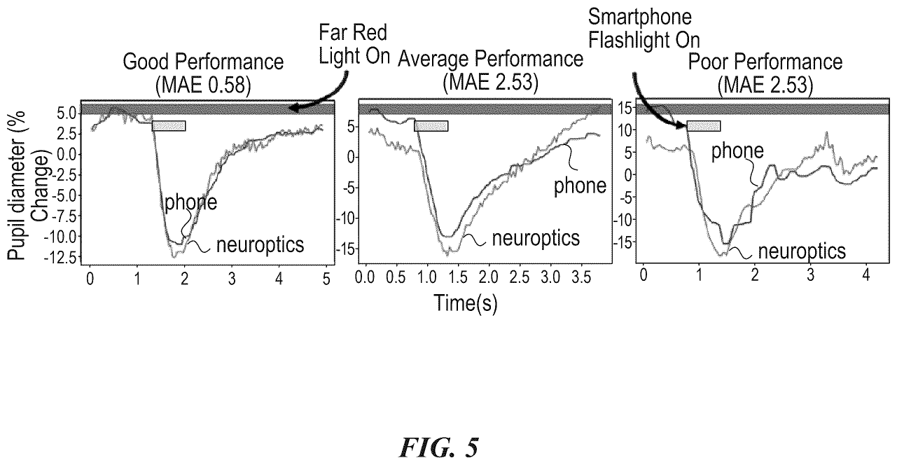

The smartphone-based pupillometer (e.g., the pupillometer attachment module 400) described above has been compared to a Neuroptics PLR 3000, a clinical gold standard for pupil measurements. The PLR test records the pupil response to a 1s flash of light. The test showcases the performance of the smartphone system during a large, fast change in pupil size. FIG. 5 illustrates pupil diameter comparison of pupillary light reflex responses recorded with the portable pupillometer device and Neuroptics pupillometer. In particular, FIG. 5 exemplifies typical performances from the prototype pupillometer attachment module and smartphone system compared to the Neuroptics pupillometer. The plots included in FIG. 5 are labelled for good, average, and poor performances representative of the system performance. Also, red and yellow shading indicate the times when the far red light and full spectrum smartphone flashlight respectively illuminate the eye. As shown, the prototype portable pupillometer device, combined with smartphone cameras, reveals a good performance when involving far red spectrum light.

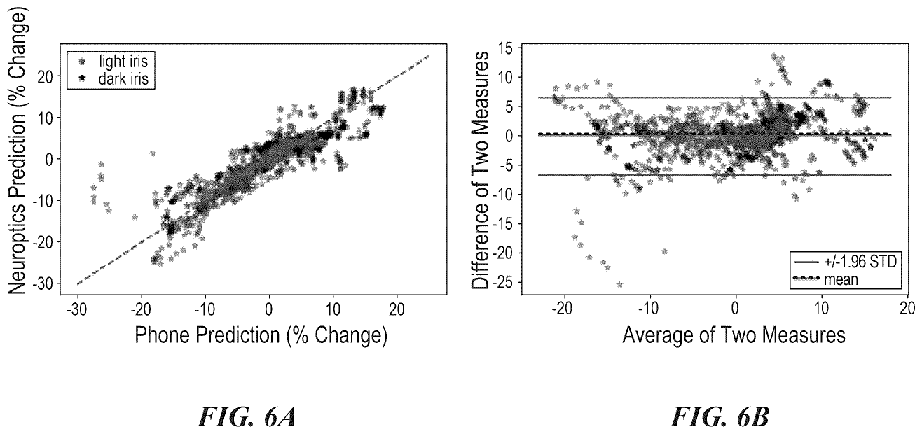

FIG. 6 provides a Regression and Bland Altman plots of pupil diameter results collected by the portable pupillometer device and Neuroptics pupillometer. Specifically, FIG. 6 demonstrates the performance of the portable pupillometer device of the present technology on N=12 participants with an average pearson's correlation coefficient of 0.93, a MAE of 2.25%, and a standard deviation of 1.3%. Note that one participant (P12) is excluded from this analysis due to errors in the Neuroptics pupillometer readings.

The performance of the novel smartphone pupillometry method on smartphone devices of different manufacturers has been compared to ensure functionality across a range of devices with different operating systems, hardware configurations, cameras, and physical form factors. In this example, the pupil-to-iris contrast is used to determine performance on the Google Pixel 4, Apple iphone 12, Samsung Galaxy A53, and Motorola Moto G Power 2022. For each phone, the pupil to iris contrast is calculated for video frames illuminated with the full visual spectrum and for pupil video frames illuminated with the target far red spectrum. Note that two participant (P11 and P13) did not participate in the cross device compatibility study. Table 2 shows the pupil to iris contrast values for all 11 participants. In this example, the average percent increase in contrast across all participants and all smartphones is 451%. In comparison, the average increase in contrast for dark brown eye participants only is 624%.

TABLE 2
Cross Phone Pupillometry Performance Comparison

| | | | | | | | Eye color | | |
|---|---|---|---|---|---|---|---|---|---|
| | | | | | | | | Iris | |
| | Pupil Iris contrast percent increase | | | | | | DSLR | Lumi- | |
| ID | A53 | Iphone12 | Moto G | Pixel4 | Mean | STD | contrast | nance | Description |
| 1 | 111.3 | 106.6 | 26.7 | 426.8 | 186.7 | 211.8 | 89 | 116 | Blue |
| 2 | 252.9 | 215.1 | 202.6 | 161.2 | 193.0 | 28.2 | 47 | 104 | Blue |
| 3 | 80.9 | 6.1 | 125.7 | 142.3 | 91.4 | 74.3 | 60 | 84 | Blue |
| 4 | 137.3 | 175.7 | 225.0 | −29.9 | 123.6 | 135.2 | 31 | 63 | Light brown |
| 5 | 500.3 | 962.6 | 1956.2 | 205.4 | 1041.4 | 878.0 | 26 | 55 | Dark brown |
| 6 | 159.2 | 154.5 | 381.2 | 131.1 | 222.2 | 138.2 | 22 | 45 | Dark brown |
| 7 | 231.1 | 103.3 | −65.7 | 277.2 | 104.9 | 171.5 | 20 | 42 | Dark brown |
| 8 | 185.7 | 941.9 | 597.2 | 107.7 | 549.0 | 419.2 | 13 | 36 | Dark brown |
| 9 | 180.6 | 310.9 | 482.6 | 349.1 | 380.9 | 90.1 | 15 | 35 | Dark brown |
| 10 | 216.7 | 396.1 | 3464.6 | 225.9 | 1362.2 | 1822.7 | 14 | 34 | Dark brown |
| 12 | 1579.2 | 134.8 | 2232.8 | −232.9 | 711.6 | 1330.2 | 9 | 26 | Dark brown |

In this example, the portable pupillometer system is designed to constantly illuminate the eye with a 625 nm light. Providing this far red light before the white light stimulus allows the participant's pupil to be adapted to the far red light such that the measurement records only the response to the white light. Unlike white light or blue light, red light does not provide a strong stimulus for pupil constriction. It has been demonstrated that red light alone causes far less pupillary constriction compared to a combined blue and red light condition. The present technology suggests that, although red light will cause some constriction of the pupil, pupil response tasks will not be significantly limited. In fact, the red light is a sufficiently weak stimulus for the pupil, such that the pupil maintains significant capacity to constrict in response to other stimuli. Also, the lighting is constant for the duration of the testing, so it does not evoke changes within the pupil during the test. As a result, pupil response test involving other visual, cognitive, or emotional stimuli can be performed without obstruction.

As shown and described on Table 2, the cross device validation compares performance of the prototype system across 4 smartphone models representative of a variety of manufacturers, operating systems, hardware configurations, and prices. The smartphone models are as follows: Google Pixel 4, Apple iphone 12, Motorola Moto G Power 2022, Samsung Galaxy A53. The cross device validation utilizes the pupil to iris contrast in recorded video frames as the key metric for comparison. This metric represents the ability to discern the pupil from the iris. The measurement is performed on all four phones for each participant. For each phone, the pupil to iris contrast in calculated in two conditions meant to compare the proposed prototype of this paper with the normal visual spectrum. Condition 1 includes the standard prototype described in 2.2 with far red LEDs and a long pass filter. Condition 2 includes the prototype without the far red LEDs or the long pass filter. For condition 2, the pupil is illuminated only with the smartphone camera covered by the neutral density filter of the prototype. Both conditions include the macro lens and the eye cup attachment.

$$Contrast \% Increase = \frac{{PupilIrisContrast}_{farred} - {PupilIrisContrast}_{full}}{\vert {PupilIrisContrast}_{full} \vert} \qquad ( 1 )$$

In computing the Pupil Iris Contrast Percent Increase in Table 2, the present technology adopts below equation, where

PupilIrisContrast=IrisPixelIntensity−PupilPixelIntensity.

The pupil iris contrast of an image is determined by comparing the light intensity of the pupil to the light intensity of the iris. This calculation is performed on video frames from each smartphone with and without the full prototype system. Video frames are converted from RGB to grayscale by selecting the red channel of the image only. The red channel contains the wavelengths of interest so it holds the majority of the signal. From the red channel, 15 by 15 blocks of 225 representative pixels are selected from the pupil and the iris for color contrast comparison. The pixels are chosen as the center pixels of the pupil. From the center of the pupil, the iris pixels are chosen from approximately 2 times the radius at an angle of 45 degrees. Adjustments to the selected pixel locations are made to avoid glare that may interfere with the calculations. The difference in pixel intensity between the pupil and iris is reported as the contrast.

In the present technology, the prototype portable pupillometer system records videos of the eye during a pupillometry test and then adopts a CNN for segmenting the pupil and iris. After the smartphone records the pupillometer video, the data can be offloaded onto a secure server for processing. The recorded video can be converted to frame by frame pupil diameter values. For example, each frame of a video can be down sampled and converted to a single channel grayscale image used as an input to a PupilLocator network with minor modifications. The CNN outputs pupil diameter and position values. Videos pupil predictions overlayed on original recorded videos are available in the supplemental material.

In this example and for the Neuroptics PLR-3000 system, the pupil diameter values are directly provided along with blink detection estimates. The Neuroptics device does not provide insight or alterations for the pupil diameter estimation methodology. From the Neuroptics data, the data points that are detected as blinks are excluded in the analysis. To avoid characterizing possible errors from the prototype system as blinks, the data points from the prototype system corresponding to the detected blinks in the Neuroptics system are excluded.

Commodity RGB cameras on smartphones provide an ideal platform for expanding the use and research of pupillometry outside of high resource clinics. The present disclosure discloses a solution to enable pupillometry measurements on RGB smartphone cameras by leveraging a novel methodology of imaging the pupil in the far red light spectrum. This methodology is validated with a prototype attachment to demonstrate the success of the new methodology, especially on individuals with dark irises. The present technology leads to a streamlined design amenable to low-cost manufacturing of pupillometry. It is to be understood that the specific embodiments disclosed herein are merely illustrative of the inventive concept, which may be embodied in various forms. Therefore, diverse modifications and refinements can be affected without departing from the concept and scope of the invention.

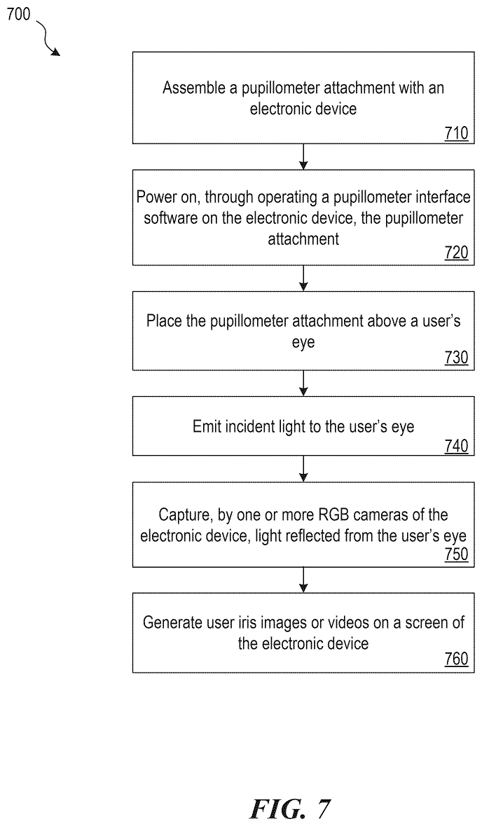

FIG. 7 is a flow chart illustrating a method 700 for operating a portable pupillometer device in accordance with various embodiments of the present technology. The method 700 includes assembling a pupillometer attachment with an electronic device, at 710. For example, the pupillometer attachment can be assembled with a smartphone, covering the RGB cameras of the smartphone for pupillometry applications, as shown in FIG. 1A.

The method 700 also includes powering on, through operating a pupillometer interface software on the electronic device, the pupillometer attachment, at 720. For example, the switch attached on the PCB 404 of the pupillometer attachment module 400 can be turned to on to power on the LEDs, as illustrated in FIGS. 4A and 4B.

In addition, the method 700 includes placing the pupillometer attachment above a user's eye, at 730. For example, a camera preview of the eye can be utilized to position the portable pupillometer device correctly above the user's eye before starting the tests. As shown in FIG. 1A, the user can position the pupil clearly within the camera frame with the pupillometer attachment module 400 (e.g., the eye cup 402) touching the test participant's face.

The method 700 further includes emitting incident light to the user's eye, at 740. For example, a far red spectrum light with wavelength ranging from 630 nm to 700 nm can be emitted from the LEDs disposed on the PCB 404, as illustrated on FIGS. 4A and 4B.

Moreover, the method 700 includes capturing, by one or more RGB cameras of the electronic device, light reflected from the user's eye, at 750. For example, the far red spectrum light reflected from the test participant's eye can be reflected, through the pupillometer attachment module 400, and received by the RGB cameras of the smartphone.

Lastly, the method 700 includes generating user iris images or videos on a screen of the electronic device, at 760. For example, as shown in FIG. 1A, the test participant's iris and pupil image or video can be displayed on the screen of the smartphone. The smartphone can records images or videos of the test participant's eye during the pupillometry test.

The scope of pupillometry measurements by the present technology holds the potential for significant extension in subsequent endeavors. The present technology focuses on the application of PLR tasks because light response tests are commonly used in both clinical and research settings. The PLR tasks demonstrate that far red light does not impede the ability of measuring the pupil response to white light stimulus. In addition, other common tasks including digit span recall tasks, oddball auditory tasks, or emotion-based tasks are also feasible with a smartphone-based pupillometry system specifically the pupillometer attachment module describe above. Another advantage of leveraging smartphone technology lies in the inherent flexibility of smartphone applications to foster innovative methodologies for delivering novel stimuli.

The smartphone-based system described in the present technology offers enhancements in user interaction and comprehensibility via application features. This system anticipates a variety of operational scenarios, each with distinct user interaction mechanisms. For instance, while the initial design anticipates an assistant executing measurements on a subject, alterations could permit self-measurement capabilities, aligning the scope with the front-facing RGB camera. Previous iterations of smartphone-based pupillometry underscore its accessibility, particularly in geriatric populations, indicating its potential for at-home deployments.

In general, pupillometry can be limited by the inability to account for eye movement. Prospective enhancements of the portable pupillometry of the present technology aim to incorporate eye-tracking functionality to mitigate the impact of ocular movement on measurement precision. This advancement not only refine the accuracy of pupillary assessments but also to initiate a new domain of eye-tracking investigations within the existing framework. It should be noted that eye-tracking algorithms can be executed independently of precise pupil delineation, with prior smartphone applications serving as a testament to this capability.

Forthcoming explorations into smartphone pupillometry utilizing the proposed methodological framework may necessitate the development of standardized procedures or calibration techniques to enhance the uniformity of measurements across different smartphone models. While the current methodology underscores the capacity for the majority of smartphones to execute PLR assessments accurately via an RGB camera, the disparity in hardware components could introduce measurement variances. For instance, deviations in the smartphone flashlight LED among models could affect the luminosity and spectral composition during stimulation, thereby altering the observed response. Environmental light conditions may also exert an influence on the pupillary reaction, as responses can vary from those recorded in brightly lit conditions to those in darkness. Addressing these diagnostic challenges and achieving measurement consistency across variable conditions and devices mandates further empirical study and potential technological innovation.

In a first example, the present disclosure describes a portable pupillometer device comprising: an electronic device having a frontside surface and a rear surface, the electronic device comprising one or more RGB cameras disposed on the frontside surface of the electronic device; a light source; and a pupillometer attachment disposed on the frontside surface of the electronic device, comprising: a case, a long-pass far red spectrum filter disposed in the case, the long-pass far red spectrum filter is configured to pass incident light in a far red spectrum ranging from 630 nm to 700 nm, and an internal opening disposed between the light source and the long-pass far red spectrum, wherein the light source is aligned internally to a periphery of the internal opening to allow incident light to be emitted through the long-pass far red spectrum, wherein the light source is configured to emit light to a direction of a user's eye through the long-pass far red spectrum filter, and the long-pass far red spectrum filter is configured to pass reflected light that is reflected from the user's eye and that has a wavelength falling in the far red spectrum, and wherein the one or more RGB cameras are aligned with a center of the internal opening to allow reflected light to be received by the one or more RGB cameras through the center of the internal opening.

In a second example, the present disclosure describes the portable pupillometer device of the first example, wherein the electronic device is a smartphone.

In a third example, the present disclosure describes the portable pupillometer device of the second example, wherein the light source is a screen of the smartphone.

In a fourth example, the present disclosure describes the portable pupillometer device of the second example, wherein the light source is a flashlight of the smartphone, the flashlight being disposed on the frontside surface or the rear surface of the smartphone.

In a fifth example, the present disclosure describes the portable pupillometer device of the fourth example, wherein the pupillometer attachment further comprises a neutral density filter that is disposed in the case and that is configured to lessen an intensity of the flashlight.

In a sixth example, the present disclosure describes the portable pupillometer device of the first example, wherein the pupillometer attachment further comprises a macro lens that is disposed above a top surface of the long-pass far red spectrum filter.

In a seventh example, the present disclosure describes the portable pupillometer device of the sixth example, wherein the macro lens has a focal length ranging from 1 mm to 50 mm and a magnification ranging from 5 to 30.

In an eighth example, the present disclosure describes the portable pupillometer device of the first example, wherein the pupillometer attachment further comprises a PCB that is disposed in the case, the PCB having a periphery region that allows light reflected from the user's eye to pass through.

In a ninth example, the present disclosure describes the portable pupillometer device of the eighth example, wherein the light source is disposed on a frontside surface of the PCB.

In a tenth example, the present disclosure describes the portable pupillometer device of the eighth example, wherein the light source comprises one or more light emitting diodes (LEDs) configured to emitting light with wavelengths ranging from 630 to 700 nm, the one or more LEDs being disposed on a frontside surface of the PCB.

In an eleventh example, the present disclosure describes the portable pupillometer device of the tenth example, further comprises a power source disposed on a backside surface of the PCB.

In a twelfth example, the present disclosure describes the portable pupillometer device of the first example, wherein the pupillometer attachment further comprises: an eye cup assembled with the case; and a mounting structure including one or more screw caps and one or more fastening screws, where in the mounting structure is attached to the case and is configured to fastening the pupillometer attachment to the electronic device.

In a thirteenth example, the present disclosure describes a portable pupillometer device, comprising: an electronic device including one or more RGB cameras disposed on a surface of the electronic device; and a pupillometer attachment assembled to the surface of the electronic device, the pupillometer attachment covers the one or more RGB cameras and comprises: a case, a custom circular PCB that is disposed in the case, the PCB having a center region and a periphery region that allow light passing through, and one or more LEDs disposed on a frontside surface of the PCB, the LEDs emitting light with wavelengths ranging from 630 to 700 nm, wherein the one or more LEDs are configured to emit a far red spectrum light with a wavelength ranging from 630 nm to 700 nm to a user's eye, and the one or more RGB cameras captures reflected far red spectrum light from the user's eye.

In a fourteenth example, the present disclosure describes the portable pupillometer device of the thirteenth example, further comprising a macro lens that is disposed at a center of the PCB, the macro lens having a focal length ranging from 1 mm to 50 mm and a magnification ranging from 5 to 30.

In a fifteenth example, the present disclosure describes the method of the thirteenth example, wherein the pupillometer attachment further comprises: an eye cup assembled with the case; and a mounting structure including one or more screw caps and one or more fastening screws, where in the mounting structure is attached to the case and is configured to fastening the pupillometer attachment to the electronic device.

In a sixteenth example, the present disclosure describes a method of operating a portable pupillometer device, comprising: assembling a pupillometer attachment with an electronic device; powering on, through operating a pupillometer interface software on the electronic device, the pupillometer attachment; placing the pupillometer attachment above a user's eye; emitting incident light to the user's eye; capturing, by one or more RGB cameras of the electronic device, light reflected from the user's eye; and generating user iris images or videos on a screen of the electronic device.

In a seventeenth example, the present disclosure describes the method of the sixteenth example, wherein assembling the pupillometer attachment with the electronic device includes: fastening, by using a mounting structure, the pupillometer attachment to the electronic device, and covering the one or more RGB cameras of the electronic device by the pupillometer attachment.

In an eighteenth example, the present disclosure describes the method of the sixteenth example, wherein emitting incident light includes stimulating a flashlight of the electronic device, and wherein the capturing of light reflected from the user's eye includes filtering or passing reflected light with wavelengths ranging from 630 nm to 700 nm by a long-pass far red spectrum filter disposed in the pupillometer attachment.

In a nineteenth example, the present disclosure describes the method of the sixteenth example, wherein emitting the incident light includes emitting, by one or more LEDs of the pupillometer attachment, a far red light with a wavelength ranging from 630 nm to 700 nm by one or more LEDs included in the pupillometer attachment, and wherein capturing the light reflected from the user's eye includes capturing the far red light reflected from the user's eye by the one or more RGB cameras of the electronic device.

In a twentieth example, the present disclosure describes describe the method of the sixteenth example, further comprises generating user pupil diameter and position values through analyzing the user iris eye images or videos by one or more convolution neural network (CNN).

It is understood that the various disclosed embodiments may be implemented individually, or collectively, using devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

Only a few implementations and examples are described and other implementations, enhancements and variations can be made based on what is described and illustrated in this patent document.

What is claimed is:

1. A portable pupillometer device, comprising:
an electronic device having a frontside surface and a rear surface, the electronic device comprising one or more RGB cameras disposed on the frontside surface of the electronic device;
a light source; and
a pupillometer attachment disposed on the frontside surface of the electronic device, comprising:
a case,
a long-pass far red spectrum filter disposed in the case, the long-pass far red spectrum filter is configured to pass incident light in a far red spectrum ranging from 630 nm to 700 nm, and
an internal opening disposed between the light source and the long-pass far red spectrum, wherein the light source is aligned internally to a periphery of the internal opening to allow incident light to be emitted through the long-pass far red spectrum,
wherein the light source is configured to emit light to a direction of a user's eye through the long-pass far red spectrum filter, and the long-pass far red spectrum filter is configured to pass reflected light that is reflected from the user's eye and that has a wavelength falling in the far red spectrum, and
wherein the one or more RGB cameras are aligned with a center of the internal opening to allow reflected light to be received by the one or more RGB cameras through the center of the internal opening.

2. The portable pupillometer device of claim 1, wherein the electronic device is a smartphone.

3. The portable pupillometer device of claim 2, wherein the light source is a screen of the smartphone.

4. The portable pupillometer device of claim 2, wherein the light source is a flashlight of the smartphone, the flashlight being disposed on the frontside surface or the rear surface of the smartphone.

5. The portable pupillometer device of claim 4, wherein the pupillometer attachment further comprises a neutral density filter that is disposed in the case and that is configured to lessen an intensity of the flashlight.

6. The portable pupillometer device of claim 1, wherein the pupillometer attachment further comprises a macro lens that is disposed above a top surface of the long-pass far red spectrum filter.

7. The portable pupillometer device of claim 6, wherein the macro lens has a focal length ranging from 1 mm to 50 mm and a magnification ranging from 5 to 30.

8. The portable pupillometer device of claim 1, wherein the pupillometer attachment further comprises a PCB that is disposed in the case, the PCB having a periphery region that allows light reflected from the user's eye to pass through.

9. The portable pupillometer device of claim 8, wherein the light source is disposed on a frontside surface of the PCB.

10. The portable pupillometer device of claim 8, wherein the light source comprises one or more light emitting diodes (LEDs) configured to emitting light with wavelengths ranging from 630 to 700 nm, the one or more LEDs being disposed on a frontside surface of the PCB.

11. The portable pupillometer device of claim 10, further comprises a power source disposed on a backside surface of the PCB.

12. The portable pupillometer device of claim 1, wherein the pupillometer attachment further comprises:
an eye cup assembled with the case; and
a mounting structure including one or more screw caps and one or more fastening screws, where in the mounting structure is attached to the case and is configured to fastening the pupillometer attachment to the electronic device.

13. A portable pupillometer device, comprising:
an electronic device including one or more RGB cameras disposed on a surface of the electronic device; and
a pupillometer attachment assembled to the surface of the electronic device, the pupillometer attachment covers the one or more RGB cameras and comprises:
a case,
a custom circular PCB that is disposed in the case, the PCB having a center region and a periphery region that allow light passing through, and
one or more light emitting diodes (LEDs) disposed on a frontside surface of the PCB, the LEDs emitting light with wavelengths ranging from 630 to 700 nm,
wherein the one or more LEDs are configured to emit a far red spectrum light with a wavelength ranging from 630 nm to 700 nm to a user's eye, and the one or more RGB cameras captures reflected far red spectrum light from the user's eye.

14. The portable pupillometer device of claim 13, further comprising a macro lens that is disposed at a center of the PCB, the macro lens having a focal length ranging from 1 mm to 50 mm and a magnification ranging from 5 to 30.

15. The portable pupillometer device of claim 13, wherein the pupillometer attachment further comprises:
an eye cup assembled with the case; and
a mounting structure including one or more screw caps and one or more fastening screws, where in the mounting structure is attached to the case and is configured to fastening the pupillometer attachment to the electronic device.

16. A method of operating a portable pupillometer device, comprising:
assembling a pupillometer attachment with an electronic device;
powering on, through operating a pupillometer interface software on the electronic device, the pupillometer attachment;
placing the pupillometer attachment above a user's eye;
emitting incident light to the user's eye;
capturing, by one or more RGB cameras of the electronic device, light reflected from the user's eye; and
generating user iris images or videos on a screen of the electronic device.

17. The method of claim 16, wherein assembling the pupillometer attachment with the electronic device includes:
fastening, by using a mounting structure, the pupillometer attachment to the electronic device, and
covering the one or more RGB cameras of the electronic device by the pupillometer attachment.

18. The method of claim 16, wherein emitting incident light includes stimulating a flashlight of the electronic device, and wherein the capturing of light reflected from the user's eye includes filtering or passing reflected light with wavelengths ranging from 630 nm to 700 nm by a long-pass far red spectrum filter disposed in the pupillometer attachment.

19. The method of claim 16, wherein emitting the incident light includes emitting, by one or more light emitting diodes (LEDs) of the pupillometer attachment, a far red light with a wavelength ranging from 630 nm to 700 nm by one or more LEDs included in the pupillometer attachment, and wherein capturing the light reflected from the user's eye includes capturing the far red light reflected from the user's eye by the one or more RGB cameras of the electronic device.

20. The method of claim 16, further comprises generating user pupil diameter and position values through analyzing the user iris eye images or videos by one or more convolution neural network (CNN).
